(19) United States
(12) Patent Application Publication
(10) Pub. No.: US 2002/0058669 A1
(43) Pub. Date: May 16, 2002

(54) PROCESS FOR THE TREATMENT OF ORGANOPHOSPHATE POISONING

(76) Inventors: HERMAN H. P. M. VAN HELDEN, BERKEL EN RODENRIJS (NL); NICOLAAS H. W. VAN XANTEN, BILTHOVEN (NL)

Correspondence Address: RONALD J. BARON, HOFFMAN & BARON, LLP, 6900 JERICHO TURNPIKE, SYOSSET, NY, 11791

(21) Appl. No.: 09/097,505

(22) Filed: Jun. 15, 1998

(51) Int. Cl. A61K 31/52; A61K 31/47

(52) U.S. Cl. 514/261; 514/262; 514/263; 514/265; 514/266

(57) ABSTRACT

The invention relates to a process for treating organophosphate poisoning in a mammal comprising the administration of an A₁ receptor adenosine agonist.

The invention relates to a process for the treatment of organophosphate poisoning.

The currently available treatment of organophosphate (OP)-poisoning (i.e. (ir)reversible inhibition of acetylcholinesterase) is mainly based on a combined administration of a cholinesterase reactivator (oxime), a muscarinie receptor antagonist (atropine) and an anticonvulsant (diazepam). Experiments with primates in the past, however, have demonstrated that such a treatment, even when carried out immediately after OP-exposure, do not rapidly restore electroencephalographic (EEG) activity and fail to prevent neuronal brain damage and postintoxication incapacitation (Dawson et al 1995, Van Helden et al 1996, Busker et al 1996, Lalleman et al 1998, Shih and McDonough 1997). Moreover, clinical experience has indicated that oximes, although designed to reactivate the inhibited acetylcholinesterase (AChE), are not always sufficiently effective as reactivators even when administered at very high dosages (Van Helden et al 1996). Furthermore, none of the oximes can be regarded as a ‘broad spectrum’ (generic) antidote, i.e., effective against all currently existing nerve agents.

Therefore, it seems justified to conclude that the therapeutic efficacy of available oximes in nerve gas poisoning is less than optimal, especially in case of rapidly ageing (a process leading to oxime-resistance) nerve agents such as soman, and seems to have reached its limits (Dawson et al 1995; Van Helden et al 1996). Therefore, new and preferably generic approaches are necessary to improve protection.

Accumulation of acetylcholine (ACh) in the synaptic cleft is generally considered as the main cause of the symptoms that ultimately lead to death in case of OP-poisoning. Thus, it is proposed that drug-induced decrease of ACh-release in brain and muscle can prevent and counteract convulsions that are a result of OP-poisoning and improve survival rate.

In accordance with the present invention, it has been found that adenosine A₁ receptor agonists are well-suited for effectuating a decrease of ACh-release in the brain and in muscles. Hence, the invention provides a process for treating organophosphate poisoning in a mammal comprising the administration of an A₁ receptor adenosine agonist.

When compared with oxime treatment, the present approach provides a generic protection, i.e. protection against all nerve agents (including soman, sarin, tabun, VX and many other AChE-inhibitors, such as insecticides and pesticides), independent of ageing of the inhibited AChE. A process according to the invention is directed towards accumulation of ACh which causes the symptoms and death in OP-poisoning. Furthermore, lower dosages than in the oxime treatment are required in order to achieve an effective treatment.

Endogenous adenosine elicits a large variety of physiological effects through interaction with cell surface adenosine receptors, which are heterogeneous (A₁, A_{2A}, A_{2B} and A₃ receptors) and widely spread throughout the body (Collis and Hourani 1993). This large variety of physiological effects elicited by adenosine provides a potential for therapeutic application of adenosine analogues.

Adenosine itself has been registered under the name of Adenocard as an anti-arrythmic drug and for controlled hypotension during aneurysm surgery, A1 adenosine agonists have been proposed as sedatives, in supraventricular tachycardia, in type II diabetes, stroke and seizures, whereas A2 adenosine agonists have been proposed as inhibitors of aggregation in thrombosis, in diagnosis of diseases in coronary arteries, in ischemia and reperfusion. Adenosine agonists for the A3 receptor have been proposed for use in certain behavorial disturbances. Other conditions for which activation of the adenosine receptors may be useful are inflammation and some pathophysiological situations, such as anxiety and panic attacks, sleep disturbances, schizophrenia, depression, epilepsy and convulsions.

Inherent to the widespread distribution of adenosine receptor subtypes is the difficulty in obtaining desirable drug actions without concomitant side effects. For example, the profound hemodynamic disturbances observed with adenosine A₁ and A_{2A} agonists have limited their use.

A new target is the inhibition of ACh-release to counteract organophosphate poisoning. This release inhibition is mediated by the A₁ adenosine receptor. Thus, in accordance with the invention, an A₁ receptor adenosine agonist is administered. In the context of the invention, an A₁ receptor adenosine agonist is an adenosine agonist which is selective for the A₁ receptor, i.e. interacts predominantly with the A₁ receptor, particularly at lower dosages.

In addition, it has been found that application of adenosine agonists with reduced intrinsic activity, i.e. partial A₁ adenosine agonists, is highly beneficial. In the content of the invention, a partial agonist is a compound that has a submaximal physiological effect at complete receptor occupancy in a certain system. It has been found that the activity of these drugs not only depends on receptor subtypes but on tissue differences (tissue selectivity) as well (kenakin 1993). This results in less pronounced cardiovascular actions and a potential increase in selectivity of action, e.g., the inhibition of ACh release in the brain.

Accordingly, in a preferred embodiment the present invention is directed to the treatment of organophosphate poisoning in mammals comprising the administration of a partial A₁ adenosine agonist. In accordance with this preferred embodiment, severe adverse effects of the treatment with respect to blood pressure and heart rate can be significantly reduced.

Particularly preferred partial A₁ adenosine agonists for use in a process according to the invention are 8-alkylamino-substituted analogues of N⁶-cyclopentyladenosine, 8-substituted adenosine, 8-substituted theophylline-7-ribose analogues, and deoxyribose analogues of N⁶-cyclopentyladenosine (CPA), N⁶-cyclohexyladenosine (CHA), N⁶-R-phenylisopropyladenosine (R-PIA) and N⁶-S-phenylisopropyladenosine (S-PIA). These adenosine agonists have a highly beneficial therapeutic window. In other words, they combine a high activity and therapeutic efficacy with a low toxicity.

Suitable examples of the class of 8-alkylamino-substituted analogues of N⁶-cyclopentyladenosine have the formula (I)

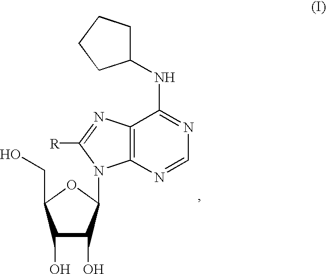

wherein R is —NHCH₃, —NHCH₄CH₃, —NH(CH₄)₄CH₃, —NH(CH₄)₃CH₃, or —NH-cyclopentyl. These compounds may be prepared in any known manner (e.g. Roelen et al 1996).

Suitable examples of the class of deoxyribose analogues of N⁶-cyclopentyladenosine (CPA), N⁶-cyclohexyladenosine (CHA), N⁶-R-phenylisopropyladenosine (R-PIA) and N⁶-S-phenylisopropyladenosine (S-PTA) have the formula (II)

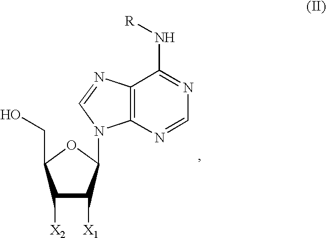

wherein R is cyclopentyl, cyclohexyl, R-phenylisopropy or S-phenylisopropyl, and wherein X₁ and X₂ are different from each other and chosen from hydrogen and hydroxyl. These compounds have been described by Van der Wenden et al 1995a.

Suitable 8-substituted adenosines and 8-substituted theophylline-7-ribose have for instance been described by Van der Wenden et al 1995b. Preferred 6-substituted adenosines have the formula (III)

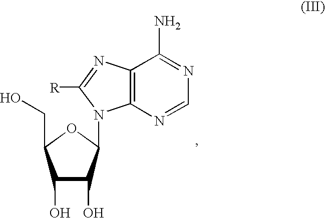

wherein R is methyl, ethyl, vinyl, thiophenyl, hydroxyl, oxymethyl, amino, aminoalkyl with from 1 to 5 carbon atoms, aminoalkylamine with from 1 to 5 carbon atoms, aminocyclopentyl, cyclohexyl, or halogen.

Preferred 8-substituted theophylline-7-riboses have the formula (IV)

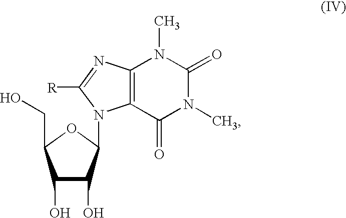

wherein R is hydrogen, amino, aminoalkyl with from 1 to 7 carbon atoms, or aminophenyl.

It will be clear that it is also possible to use suitable combinations of the above A₁ adenosine agonists for treating an OP-poisoning. A treatment comprising such a combined administration of A₁ adenosine agonist is also encompassed by the invention.

As has been mentioned above, one of the great advantages of a process according to the invention is that it is a generic process. This means that it can be used to treat organophosphate poisoning resulting from substantially all nerve agents, such as soman, sarin, tabun, VX and so forth, as well as other AChE-inhibitors, such as a large number of insecticides and pesticides. An organophosphate poisoning has been found to be based on the inhibition of the enzyme acetylcholinesterase (AchE). Inactive AChE cannot hydrolyze acetycholine (Ach) which will accumulate in the cholinergic synaps and as a result will paralyze the synaptic transmission. Apparent, outward symptoms are salivation, convulsions and respiratory paralysis.

The treatment of the invention can be applied to any mammal suffering from the effects of an OP-poisoning. However, it will be mostly applied to primates, in particular to humans.

As the effects of an OP-poisoning can be lethal within a very short period of time after the intoxication, i.e. the exposure to the poisonous compound(s), it is preferred that the treatment according to the invention is performed as soon as possible after said exposure. Desirably, the administration of an A₁ receptor adenosine agonist in accordance with the invention is carried out within one minute after acute intoxication or on guidance of symptoms. First mild symptoms are fatigue, headache, dizziness, numbness of extremities, nausea and vomiting, sweating, extreme salivation, diarrhoea, abdominal pain, frequent urination, and miosis. Moderate symptoms are generalized weakness, speech impediment, fasciculations, trembling, miosis, hampered motoric fasciculations, fever, tightness in the chest, involuntary urination and defecation.

The dosage in which the A₁ adenosine agonist can suitably be administered may vary within the range of 0.1-20 mg/kg, but is highly dependent on efficacy and adverse effects. Preferably, the dosage is chosen within the range of 1-2 mg/kg.

Effective manners in which the A₁ adenosine agonist may be administered include intramuscular, intravenous, and intranasal administration. The most preferred manners of administration are intramuscular and intravenous administration since after these application routes the A₁ adenosine agonist reaches the site of the A₁ receptor, where it is intended to effect a decrease of ACh-release, very fast.

For the above manners of administration, the A₁ adenosine agonist can most suitably be formulated in the form of a saline solution. However, in case the A₁ adenosine agonist appears insufficiently soluble in water, it may be useful to formulate them in DMSO or ethanol, diluted with a solution of sodium chloride in water (saline) to a final 10 to 30 vol. % DMSO solution, or a 5-10 vol. % ethanol solution.

OP-poisoning will be mostly encountered by people under harsh conditions, e.g. soldiers at war, anti-terrorist squads, and so forth. Moreover, it is of great importance that the treatment in accordance with the invention is performed as soon as possible after exposure to the poison. For these reasons, it is highly preferred to use a so-called (auto-injector) device for the administration of the drug. This device has for instance been developed by Astra Tech AB, Mölndal, Sweden and by Meridian Medical Technologies, Columbia, Md., USA. In order to administer an A₁ receptor adenosine agonist using an auto-injector, the auto-injector is put on a muscle (e.g. a thigh muscle), and after pressing a button, a hollow needle penetrates the skin into the muscle and a unit-dose of the desired A₁ receptor adenosine agonist is injected into the muscle. Thus, the invention also encompasses an auto-injector holding a formulation comprising an A₁ receptor adenosine agonist as disclosed hereinabove.

The invention will now be elucidated by the following, non-restricting examples.

### EXAMPLE I

An experiment was carried out using 5′-N-ethylcarboxamido-adenosine (NECA), an agonist of A₁ and A₂ receptors, to treat OP-poisoning, using the nerve agent soman as an irreversible model AChE-inhibitor.

Based on in vitro experiments in which 300 nM of NECA appeared to decrease the release of ACh from a rat diaphragm endplate zone by 70%, NECA was tested in an in vivo experiment. The in vitro concentration was extrapolated to an in vivo dose. Assuming that NECA distributes homogeneously in the body and that 300 nM is the effective concentration in the brain (300 nM=300 nMol/lit/kg≈0.1 mg/kg i.m.), 0.1 mg/kg of NECA was administered intramuscularly (i.m.). In a practical protocol, this calculated dose was administered intramuscularly at 1 min following a subcutaneous poisoning with 1.5 or 2 LD₅₀ soman in unanaesthesized rats. Symptoms and survival were registered. The results of this pilot are presented in Table 1 and show that 0.1 mg/kg (i.m.) NECA prevents and postpones the appearance of convulsive activity, and tends to improve the survival rate.

### EXAMPLE II

In this experiment cyclopentyl adenosine (CPA), a highly specific A₁ adenosine receptor agonist, was tested using a similar protocol as described in Example I. The therapeutic efficacy of two doses (1 and 2 mg/kg, i.m.) of the latter compound was tested against 1.9 LD₅₀ soman in a similar way as described in Example I for NECA. The results are presented in Table 2 showing that administration of CPA prevented convulsions and led to survival of each animal in a healthy condition judging from clinical observation.

### EXAMPLE III

In separate experiments, using a limited number of unanaesthesized rats, it was investigated to what degree CPA (2 mg/kg i.m.) would be able to prevent the accumulation of ACh in the striate body (corpus striatum) of the brain upon soman poisoning (2 LD₅₀ s.c.) by performing brain microdialysis according to the method described by Moor et al (1994). Briefly, rats were anaesthetized with chloral hydrate (400 mg/kg i.p.) and a dialysis probe was implanted in the striate body. The actual dialysis experiments were conducted around 20 h after surgery.

Probes were perfused with artificial cerebrospinal fluid containing in mM; NaCl 147, KCl 3.0, CaCl₂ 1.2, and MgCl₂ 1.2. The AChE-inhibitor neostigmine bromide (10⁻⁸ M) was added to this perfusion fluid to obtain detectable quantities of base-line ACh. The artificial cerebrospinal fluid was delivered by a syringe pump (Carnegy Medicine, Sweden) at a rate of 2 μl/min. Ten min samples were collected in a 50-μl loop of an injection valve that was automatically activated by an electronic timer.

After stabilisation of the ACh levels, soman (1-2 LD₅₀) was injected subcutaneously followed by intramuscular injection of CPA (2 mg/kg) 1 minute later. These preliminary results demonstrated a low level of extracellular brain ACh (0-50 fold increase in ACh) following CPA treatment of soman poisoning, which was in contrast to a large increase in the amount of extracellular ACh in soman-poisoned animals not treated with CPA (180 400 fold increase in ACh). This low level of ACh-release in the brain following soman poisoning and CPA treatment was associated with postponement or lack of symptoms, and survival of the animals. Soman poisoned animals (controls) showed convulsions and died within 20 minutes.

### EXAMPLE IV

A number of partial A₁ receptor agonists was tested in a similar way as described for NECA and CPA in Examples I and II. Advantageous therapeutic efficacy against soman and carin in rats and guinea pigs was demonstrated while the adverse effects on blood pressure and heart rate were less than in case of NECA and CPA.

The protocol in which the efficacy of the partial A₁ receptor agonists against AChB inhibitors was tested, was not standard; both repetitive- and prophylactic administration (intramuscularly or intravenously) were investigated. Neither the level of intoxication was standard; it was in the range of 0.5-3 LD₅₀ of AChE-inhibitors tested.

TABLE 1
Protection of NECA (0.1 mg/kg intramuscularly)
injected 1 min. after soman-poisoning in rats (n indicates
the number of animals tested).

| Treatment | Survival time | Symptoms |
|---|---|---|
| Soman (n = 5) | <15 min. | starting at 5-7 min.: |
| (2 LD₅₀ s.c.) | | salivation, convulsions, |
| | | respiratory distress |
| Soman (2 LD₅₀ | >24 h; | normal respiration; |
| s.c) + NECA | >24 h; | alert, dry mouth, drank water; |
| (n = 5) | 33 min.; | convulsions at t = 21 min.; |
| | 90 min.; | convulsions at t = 50 min,; |
| | 75 min. | convulsions at t − 40 min. |
| Soman (1.5 | >24 h | starting at 2-10 min.; |
| LD₅₀ s.c) | | convulsive activity for hours |
| (n = 3) | | on end |
| Soman (1.5 | >24 h | no convulsions at all |
| LD₅₀ s.c.) + | | |
| NECA | | |
| (n = 3) | | |
| NECA (n = 2) | >24 h | no physical signs |

TABLE 2
Protection of CPA (intramuscular) injected 1 min.
after soman-poisoning (1.9 LD₅₀ s.c.) in rats (n indicates
the number of animals tested).

| Treatment | Survival time | Symptoms |
|---|---|---|
| Soman (n = 6) | 17-28 min. | after 5-7 min.: chewing, |
| | | salivation, convulsions, |
| | | respiratory distress |
| Soman + CPA | >24 h | 3 rats: no symptoms; |
| (1 mg/kg) | | 1 rat: chewing after 32 min., |
| (n = 6) | | then salivation, convulsions |
| | | and decreased respiration; |
| | | 1 rat: decreased respiration |
| | | frequently, and salivation |
| | | next morning; |
| | | 1 rat: chewing after 10 min. |
| Soman + CPA | >24 h | 5 rats: no symptoms, normal |
| (2 mg/kg) | | respiration, alert, dry mouth, |
| (n = 6) | | drank water; |
| | | 1 rat: some traces of blood |
| | | around mouth next morning. |
| CPA (2 mg/kg) | >24 h | no physical signs |
| (n = 2) | | |

References

*Busker R W, Zijlstra J J, Philippens, Groen B, Melchers S P C (1996) Comparison of the efficacy of single or repeated HI-6 treatment following soman poisoning in guinea pigs and marmoset monkeys, Toxicol 112: 183-194

*Collis M G and Hourani S M O (1993) Adenosine receptor subtypes, TiPS 14: 360-366

*Correio de-Sa P, Sebastiao A M, Ribeiro J A (1991) Inhibitory and excitatory effects of adenosine receptor agonists on evoked transmitter release from phrenic nerve ending of the rat, Br J. Pharmacol 103 921: 1614-1620

*Dawson R M (1994) Review of oximes available for treatment of nerve agent poisoning, J Appl Toxicol 14:317-31

*Kenakin T P (1993) Pharmacological analysis of drug receptor interaction, pp 441-468, Raven Press, New York

*Lallement G, Clarencon D, Masqueliez C, Baubichon D, Galonnier M, Burckhart M-F, Peoc'h M, Mestries J-C (1998) Nerve agent poisoning in primates: antilethal, anti-epileptic and neuroprotective effects of GK-11. Arch Toxicol 72:84-92

*Moor E, De Boer P, Beldhuis H J A, Westerink B H C (1994) A novel approach for studying septo-hippocampal cholinergic neurons in freely moving rats: a microdialysis study with dual-probe design. Brain Res 648: 32-38

*Roelen H, Veldman N, Spek A L, Von Frijtag Drabbe Kunzel J K, Mathot R A A, IJzerman A P (1996) N6, C8-disubstituted adenosine derivatives as partial agonist for adenosine A1 receptors. J Med Chem 39: 1463-1471

*Sebastiao A M, Stone T W, Ribeiro J A (1990) The inhibitory adenosine receptor at the neuromuscular junction and hippocampus of the rat: antagonism by 1,3,8-substituted xanthines, Br J Pharmacol 101, 453-459

*Shih T M, McDonough J H (1997) Neurochemical mechanisms in soman-induced seizures. J Appl Toxicol 17: 255-264

*Van der Wenden E M, Von Frijtag Drabbe Kunzel J K, Mathot R A A, Danhof M, IJzerman A P, Soudijn W (1995a) Ribose-modified adenosine analogs as potential partial agonists for the adenosine receptor, J Med Chem 38: 4000-4006

*Van der Wenden E M, Hartog-Witte H R, Roelen H C P F, Von Frijtag Drabbe Kunzel J K, Pirovano I M, Mathot R A A, Danhof M, Van Aerschot A, Lidaks M J, IJzerman A P, Soudijn W (1995b) 8-Substituted adenosine and theophylline-7-riboside analogs as potential partial agonists for the adenosine A1 receptor. Eur J Pharmacol Mol Pharmacol Sect 290: 189-199

*Van Helden H P M, Busker R W, Melchers B P C, Bruijnzeel P L B (1996) Pharmacological effects of oximes: how relevant are they? Arch Toxicol 70:779-786

*Van Schaick E A, Mathot A A, Gubbens-Stibbo J M, Langomeijer M W E, Roelen H C P F, IJzerman A P, Danhof M (1997) 8-Alkylamino-substituted analogs of N6-cyclopentyladenosine are partial agonists for the cardiovascular adenosine A1 receptors in vivo, J Pharmacol Exp Ther 283: 800-808

1. A process for treating organophosphate poisoning in a mammal comprising the administration of an A₁ receptor adenosine agonist.

2. A process according to claim 1, wherein a partial A₁ receptor adenosine agonist is administered.

3. A process according to claim 2, wherein the partial A₁ receptor adenosine agonist is chosen from the group of 8-alkylamino substituted analogues of N⁶-cyclopentyladenosine, 8-substituted adenosine, 8-substituted theophylline-7-ribose analogues, and deoxyribose analogues of N⁶-cyclopentyladenosine (CPA), N⁶-cyclohexyladenosine (CHA), N⁶-R-phenylisopropyladenosine (R PIA) and N⁶-S-phenylisopropyladenosine.

4. A process according to claim 3, wherein the partial A₁ adenosine agonist is a 8-alkylamino-substituted analogue of N⁶-cyclopentyladenosine having the formula (I) 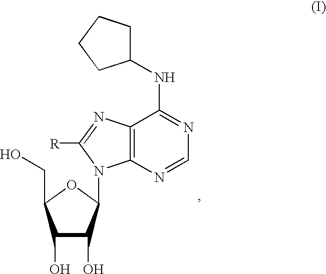
wherein R is —NHCH₃, —NHCH₂CH₂, —NH(CH₂)₃CH₃, —NH(CH₂)₃CH₃, or —NH-cyclopentyl.

5. A process according to claim 3, wherein the partial A₁ adenosine agonist is a deoxyribose analogue of N⁶-cyclopentyladenosine (CPA), N⁶-cyclohexyladenosine (CHA), N⁶-R-phenylisopropyladenosine (R-PIA) or N⁶-S-phenylisopropyladenosine having the formula (II) 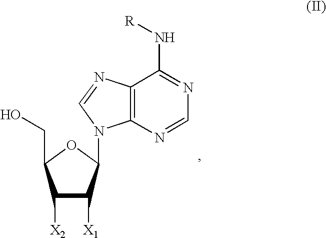
wherein R is cyclopentyl, cyclohexyl, R-phenylisopropyl, or S-phenylisopropyl, and wherein X₁ and X₂ are different from each other and chosen from hydrogen and hydroxyl.

6. A process according to claim 3, wherein the partial A₁ adenosine agonist is an 8-substituted adenosine having the formula (III) 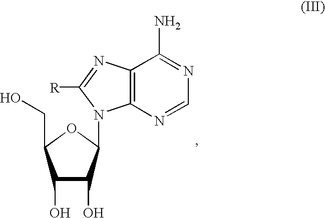
wherein R is methyl, ethyl, vinyl, thiophenyl, hydroxyl, methyoxy, amino, aminoalkyl with from 1 to 5 carbon atoms, aminoalkylamino with from 1 to 5 carbon atoms, aminocyclopentyl, cyclohexyl, or halogen.

7. A process according to claim 3, wherein the partial A₁ adenosine agonist is an 8-substituted theophylline-7-ribose having the formula (IV) 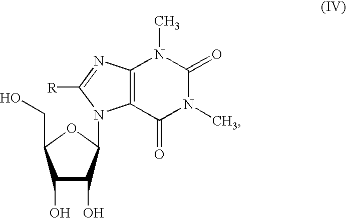
wherein X is hydrogen, amino, aminoalkyl with from 1 to 7 carbon atoms, or aminophenyl.

8. A process according to any of the preceding claims, wherein a human is treated for organophosphate poisoning.

9. A process according to any of the preceding claims, wherein the A₁ receptor adenosine agonist is administered in a dosage of 0.1-20 mg/kg.

10. A process according to any of the preceding claims, wherein the A₁ receptor adenosine agonist is administered intramuscularly or intravenously.

11. A process according to claim 10, wherein the A₁ receptor adenosine agonist is administered in the form of a saline solution.

12. A process according to claim 11, wherein the saline solution further comprises 10-30 vol. % dimethylsulfoxide.

13. A process according to claim 11 or 12, wherein the saline solution further comprises 5-10 vol. % ethanol.

14. A process according to any of the preceding claims, wherein the A₁ receptor adenosine agonist is administered by use of an injector, preferably an auto-injector.

15. An injector, preferably an auto-injector, comprising an A₁ receptor adenosine agonist.

16. Use of an A₁ receptor adenosine agonist for preparing a medicament for treating organophosphate poisoning in mammals.
